(19) United States
(12) Patent Application Publication
(10) Pub. No.: US 2021/0020313 A1
(43) Pub. Date: Jan. 21, 2021

(54) VISUALISING CLINICAL AND DISEASE DATA

(71) Applicant: GARVAN INSTITUTE OF MEDICAL RESEARCH, New South Wales (AU)

(72) Inventor: Tudor GROZA, New South Wales (AU)

(73) Assignee: GARVAN INSTITUTE OF MEDICAL RESEARCH, New South Wales (AU)

(21) Appl. No.: 16/979,775

(22) PCT Filed: Mar. 12, 2019
(86) PCT No.: PCT/AU2019/050221

§ 371 Date: Sep. 10, 2020

(30) Foreign Application Priority Data
Mar. 13, 2018 (AU) 2018201783

(51) Int. Cl. G16H 50/20; G06F 3/048; G06K 9/62; G16H 50/70

(52) U.S. Cl. CPC G16H 50/20; G16H 50/70; G06K 9/6215; G06F 3/048

(57) ABSTRACT

This disclosure relates to generating interactive graphical visualisations of clinical and data. A processor calculates phenotype-to-phenotype similarity value indicative of a similarity between observed phenotypes and each phenotype in a set of stored phenotypes based on an ontology of phenotypes. The processor then determines an assignment of stored phenotypes to observed phenotypes based on the similarity values and further aggregates the similarity values into a set-to-set similarity value indicative of a similarity between the observed phenotypes and the set of stored phenotypes. The processor then repeats these steps to calculate a set-to-set similarity measure for each of the multiple sets. Finally, the processor selects one or more of the multiple sets based on the set-to-set similarity value and generates a graphical user interface comprising a graphical indication of the selected one or more of the multiple sets in relation to the multiple observed phenotypes.

### CROSS-REFERENCE TO RELATED APPLICATIONS

The present application claims priority from Australian Patent Application No 2018201783 filed on 13 Mar. 2018, the content of which is incorporated herein by reference.

### TECHNICAL FIELD

This disclosure relates to generating interactive graphical visualisations of clinical and data.

### BACKGROUND

Clinicians generally examine patients and record their observations (phenotypes). Clinicians also have access to a stock of knowledge from specialists and researchers around the world. However, it is still difficult for clinicians to use this information efficiently. In particular, it is difficult for a clinician to decide which disorders are indicated by the currently observed phenotype.

More particularly, each disease can be defined by a set of phenotypes that are stored in large databases. However, in most cases there is not an exact match between the observed phenotypes and the phenotypes stored for a particular disorder. This makes it difficult for the clinician to explore the disorders that are most relevant for this particular patient.

For example, the Database Online Mendelian Inheritance in Man (OMIM) comprises about 7,500 disorders, which are annotated with phenotypes, where each disorder is associated with about 2-30 phenotypes. For multiple observed phenotypes it therefore quickly becomes impossible to find the most relevant disorders. Even a computer-aided approach would quickly become impractical due to excessive computational complexity and resulting slow response time.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### SUMMARY

There is a need for a computerised tool that the clinician can use and that provides access to the vast amount of data and knowledge that is available. This tool may filter the available options based on the observed phenotype so that the clinician can ultimately find a most relevant disorders.

Disclosed herein is a method that quantifies the similarity between a set of observed phenotypes and a set of stored phenotypes. This set of stored phenotypes may be characterising a disorder or may contain the phenotypes observed on another patient. A quantification of the similarity allows the sorting of candidate diseases (or sets of phenotypes), which allows the reduction of data that is to be provided to a human user. This way, the human user is able to understand the data. For example, the most similar disorders or sets of stored phenotypes may be automatically selected, which allows easy visual inspection of the different associations.

A method for creating a graphical visualisation of clinical data comprises:

receiving the clinical data indicative of multiple observed phenotypes of a patient;

accessing a set of stored phenotypes;

accessing on a database an ontology of phenotypes including hierarchical relationships between the phenotypes of the ontology;

calculating a phenotype-to-phenotype similarity value indicative of a similarity between each of the observed phenotypes and each phenotype in the set of stored phenotypes, based on the ontology;

determining an assignment of one stored phenotype of the set to each of the observed phenotypes based on the phenotype-to-phenotype similarity values;

aggregating the phenotype-to-phenotype similarity values of the stored phenotypes from the set that are assigned to each of the multiple observed phenotypes into a set-to-set similarity value indicative of a similarity between the observed phenotypes and the set of stored phenotypes;

repeating the accessing, calculating, determining the assignment and aggregating steps for each of the multiple sets of stored phenotypes to thereby calculate a set-to-set similarity measure for each of the multiple sets;

selecting one or more of the multiple sets based on the aggregated set-to-set similarity values; and

generating a graphical user interface comprising a graphical indication of the selected one or more of the multiple sets in relation to the multiple observed phenotypes.

It is an advantage that the similarity between the observed phenotypes and the sets of phenotypes is determined based on the distance in the ontology. This way, inexact matches can be considered and the candidate diseases can be selected for the clinician. Further, determining an assignment enables the use of computationally efficient heuristic algorithms which reduce the time required for computation. Together with the use of an ontology this allows rapid calculations leading to an enhanced user experience. For example, the clinician can select different patients and different disorders and immediately receive a selection of most relevant candidates without having to wait for complex calculations to be completed.

Determining the assignment may comprise determining an assignment by optimising a cost that is based on the phenotype-to-phenotype similarity values.

Determining the assignment may comprise applying a heuristic to determine the assignment by selecting one assignment at a time with optimal cost and then determining remaining assignments.

Determining the assignment may comprise performing an Hungarian algorithm.

Aggregating the phenotype-to-phenotype similarity values may comprise calculating an average of the phenotype-to-phenotype similarity values.

The method may further comprise splitting observed phenotypes and stored phenotypes by anatomical systems and aggregating set-to-set similarity values across the anatomical systems.

Aggregating across the anatomical systems may comprise calculating an average of the set-to-set similarity values the anatomical systems.

Generating the user interface may comprise generating a graphical indication of the phenotype-to-phenotype similarity values.

The graphical indication of the phenotype-to-phenotype similarity value may comprise a line with a first visual appearance for an exact match and a second visual appearance for an inexact match.

The set of stored phenotypes may be associated with a disorder.

The set of stored phenotypes may be associated with a further patient.

Calculating the phenotype-to-phenotype similarity value may comprise determining a distance in the ontology from the observed phenotype to each phenotype in the set of stored phenotypes.

Calculating the phenotype-to-phenotype similarity value may be based on an information content of the observed phenotype in the ontology and an information content of the stored phenotype in the ontology and an information content of a least common subsumer of the observed phenotype and the stored phenotype in the ontology.

The information content may be based on a count of leaf nodes under children of the phenotype in the ontology, a count of ancestors of the phenotype in the ontology and the total number of leaf nodes in the ontology.

A computer system for creating a graphical visualisation of clinical data comprises:

a data port to receive the clinical data indicative of multiple observed phenotypes of a patient;

a data store from which to access a set of stored phenotypes;

database to store an ontology of phenotypes including hierarchical relationships between the phenotypes of the ontology;

a processor to:
calculate a phenotype-to-phenotype similarity value indicative of a similarity between each of the observed phenotypes and each phenotype in the set of stored phenotypes based on the ontology;
determine an assignment of one stored phenotype of the set to each of the observed phenotypes based on the phenotype-to-phenotype similarity values;
aggregate the phenotype-to-phenotype similarity values of the stored phenotypes from the set that are assigned to each of the multiple observed phenotypes into a set-to-set similarity value indicative of a similarity between the observed phenotypes and the set of stored phenotypes;
repeat the accessing, calculating, determining the assignment and aggregating steps for each of the multiple sets of stored phenotypes to thereby calculate a set-to-set similarity measure for each of the multiple sets;
select one or more of the multiple sets based on the aggregated set-to-set similarity values; and
generate a graphical user interface comprising a graphical indication of the selected one or more of the multiple sets in relation to the multiple observed phenotypes.

Optional features described of any aspect of method, computer readable medium or computer system, where appropriate, similarly apply to the other aspects also described here.

### BRIEF DESCRIPTION OF DRAWINGS

An example will be described with reference to:

FIG. 1 illustrates a computer system for creating an interactive graphical visualisation of clinical data.

FIG. 2 illustrates a method for creating an interactive graphical visualisation of clinical data.

FIG. 3 illustrates an example ontology graph comprising an observed phenotype and a stored phenotype.

FIG. 4 illustrates a matrix of similarity values for observed phenotypes across stored phenotypes.

FIG. 5 illustrates the result of the Hungarian assignment algorithm for the matrix in FIG. 4.

FIG. 6 illustrates an example user interface comprising a graphical indication of a first set, a second set and a third set in relation to observed phenotypes.

### DESCRIPTION OF EMBODIMENTS

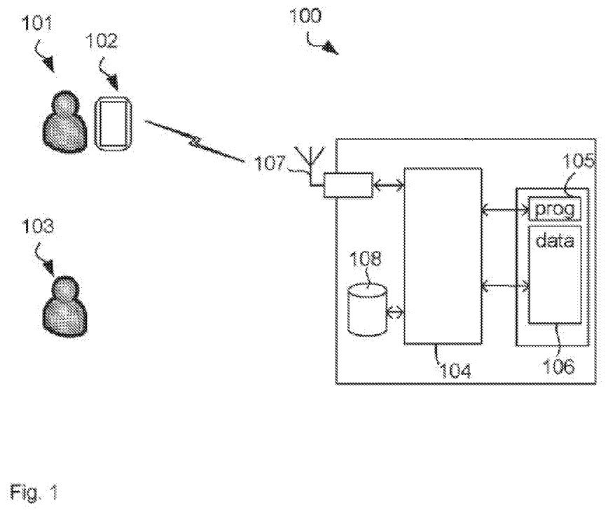

FIG. 1 illustrates a computer system 100 for creating an interactive graphical visualisation of clinical data. In one example, computer system 100 is a cloud-based computer system that is operated by clinician 101 with the use of a client device 102 such as a personal computer, tablet or other computing device. However, the proposed solution may equally be implemented locally. Clinician 101 examines a patient 103 and records clinical data into client device 102. The clinical data includes multiple phenotypes that clinician 101 observes in patient 103. It is noted that in this context phenotypes are not disorders but observations. Disorders on the other hand are conclusions that could be drawn based on the observed phenotypes.

The computer system 100 comprises a processor 104 connected to program memory 105, data memory 106, a communication port 107 and a database 108. When reference is made herein to a database, it is to be understood as any form of structured data storage including comma separated values, SQL or graph based databases, which are preferred due to their inherent ability to efficiently store and retrieve graph data as used herein.

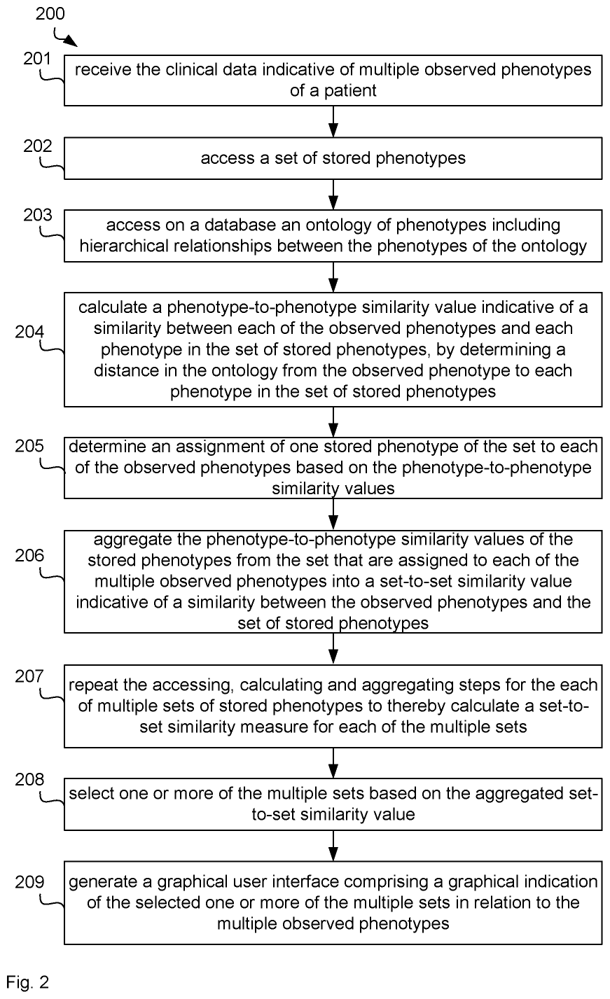

The program memory 105 is a non-transitory computer readable medium, such as a hard drive, a solid state disk or CD-ROM. Software, that is, an executable program stored on program memory 105 causes the processor 104 to perform the method in FIG. 2, that is, processor 104 receives the clinical data from client device 102, accesses database 108, determines similarity values between observed phenotypes and sets of stored phenotypes and finally creates a user interface of these similarity values.

The processor 104 may then store the graphical user interface on data store 106, such as on RAM or a processor register. Processor 104 may also send the graphical user interface via communication port 107 to client device 102 such as through the use of a web server installed on computer system 100 and a browser application installed on client device 102.

The processor 104 may receive data, such as clinical data, from data memory 106 as well as from the communications port 107. In one example, the processor 104 receives clinical data from client device 102 via communications port 107, such as by using a Wi-Fi network according to IEEE 802.11. The Wi-Fi network may be a decentralised ad-hoc network, such that no dedicated management infrastructure, such as a router, is required or a centralised network with a router or access point managing the network.

In one example, processor 104 receives and processes the clinical data in real time. This means that the processor 104 creates the graphical user interface every time clinical data is received from client device 102 and completes this step before the client device 102 sends the next clinical data update. The same may apply for re-arranging the graphical user interface such that the time between the user interacting with the graphical user interface and the graphical user interface being updated on client device 102 is not perceived as a delay, such as less than 1 s or less than 100 ms. User interaction may comprise selection of sets of stored phenotypes, such as sets associated with further patients or sets associated with disorders of interest.

Although communications port 107 is shown as distinct module, it is to be understood that any kind of data port may be used to receive data, such as a network connection, a memory interface, a pin of the chip package of processor 104, or logical ports, such as IP sockets or parameters of functions stored on program memory 104 and executed by processor 104. These parameters may be stored on data memory 106 and may be handled by-value or by-reference, that is, as a pointer, in the source code.

The processor 104 may receive data through all these interfaces, which includes memory access of volatile memory, such as cache or RAM, or non-volatile memory, such as an optical disk drive, hard disk drive, storage server or cloud storage. The computer system 100 may further be implemented within a cloud computing environment, such as a managed group of interconnected servers hosting a dynamic number of virtual machines.

It is to be understood that any receiving step may be preceded by the processor 104 determining or computing the data that is later received. For example, the processor 104 determines clinical data and stores the clinical data in data memory 106, such as RAM or a processor register. The processor 104 then requests the data from the data memory 106, such as by providing a read signal together with a memory address. The data memory 106 provides the data as a voltage signal on a physical bit line and the processor 104 receives the clinical data via a memory interface.

It is to be understood that throughout this disclosure unless stated otherwise, nodes, edges, graphs, solutions, variables, paths, sets and the like refer to data structures, which are physically stored on data memory 106 or processed by processor 104. Further, for the sake of brevity when reference is made to particular variable names, such as “similarity value” or “distance” this is to be understood to refer to values of variables stored as physical data in computer system 100.

FIG. 2 illustrates a method 200 as performed by processor 104 for creating a graphical visualisation of clinical data. FIG. 2 is to be understood as a blueprint for the software program and may be implemented step-by-step, such that each step in FIG. 2 is represented by a function in a programming language, such as C++ or Java. The resulting source code is then compiled and stored as computer executable instructions on program memory 105.

It is noted that for most humans performing the method 200 manually, that is, without the help of a computer, would be practically impossible. Therefore, the use of a computer is part of the substance of the invention and allows using the available data that would otherwise not be possible or prohibitively difficult due to the large amount of data and the large number of calculations that are involved.

FIG. 2 illustrates a method for creating an interactive graphical visualisation of clinical data as performed by processor 104. Method 200 commences by receiving 201 the clinical data. The clinical data is indicative of multiple observed phenotypes of patient 103 as entered by clinician 101 into client device 102, for example. Processor 104 also accesses 202 a set of stored phenotypes such as sets of phenotypes that characterise a disorder or sets of phenotypes that have been recorded previously for different patients potentially by different clinicians. A problem is that different clinicians often record similar observations as different phenotypes. For example, “enlarged bones”, “big bones” and “huge bones” may all define the same observation in different words. Further, some observations may be related although they appear very different. For example, “enlarged bones” and “brittle bones” both relates to anomalies of the skeletal system but this relationship is difficult to consider by clinician 101.

In order to address this issue, processor 104 accesses 203 on database 108 an ontology of phenotypes including hierarchical relationships between the phenotypes of the ontology. While database 108 is shown as integral part of computer system 108, it may equally be hosted externally, such as on a publicly available cloud computing environment. In one example, clinician 101 enters observations as text in natural language and a natural language processor analyses the text input and maps it to a phenotype ontology, such as phenotypes included in OMIM or to the Human Phenotype Ontology (http://human-phenotype-ontology.github.io, HPO). As described on their website the HPO is a computational representation of a domain of knowledge based upon a controlled, standardized vocabulary for describing entities and the semantic relationships between them.

The HPO aims to provide a standardized vocabulary of phenotypic abnormalities encountered in human disease. Each term in the HPO describes a phenotypic abnormality, such as atrial septal defect. The HPO is currently being developed using the medical literature, Orphanet, DECIPHER, and OMIM. HPO currently contains approximately 11,000 terms (still growing) and over 115,000 annotations to hereditary diseases. The HPO also provides a large set of HPO annotations to approximately 4000 common diseases.

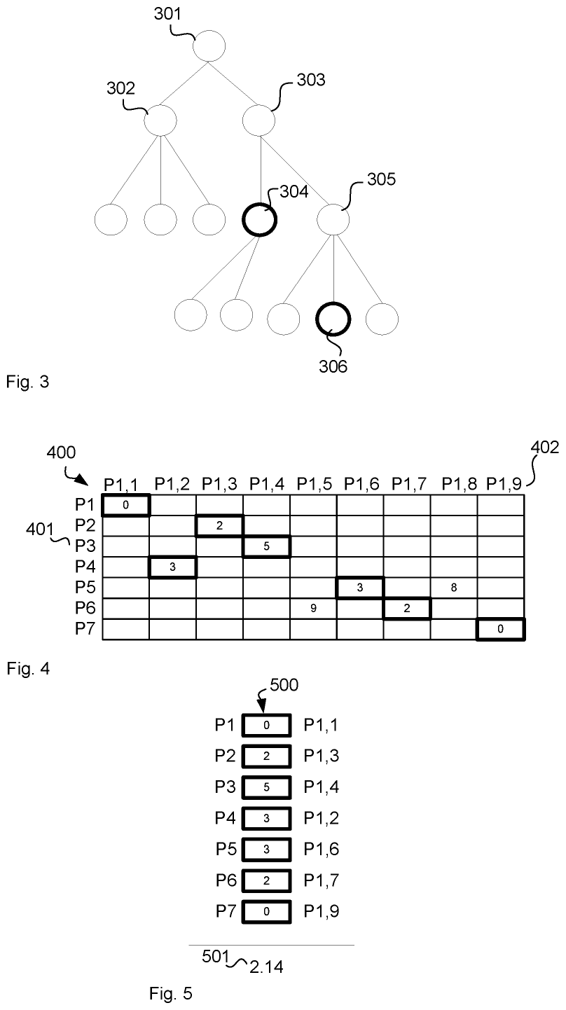

FIG. 3 illustrates an example tree graph that is illustrative of the used ontology 300. Each node (circle) represents a phenotype that can be observed and recorded by clinician 101. Some but not all nodes are labelled for ease of explanation. Root node 301 in this example represents all phenotypes of a particular physiological or anatomical system, such as anomaly of the skeletal system or anomaly of the intestines. Edges between nodes represent relationships in the sense that the lower nodes are more specific phenotypes. For example, the phenotype “enlarged head” would be a specialisation of “anomaly of the skeletal system”. However, “enlarged head” may not be connected to “anomaly of the skeletal system” by an edge directly as the intermediate generalisation of “anomaly of the head” may lie between them. In this example, an observed phenotype 304 as well as a stored phenotype 306 are marked in bold (respective nodes in FIG. 3).

Processor 104 calculates 204 a phenotype-to-phenotype similarity value indicative of a similarity between the observed phenotype 304 and a stored phenotype 306. Processor 104 performs this calculation by determining a distance in the ontology 300 from the observed phenotype 304 to the stored phenotype 306. The distance from observed phenotype 304 to stored phenotype can be computed as the distance from the root 301 to the observed phenotype 304, plus the distance from the root to the stored phenotype 306, minus twice the distance from the root to their lowest common ancestor, which would be node 303 in this case. Therefore, the distance in this case would be 2+3-2*1=3. Further details can be found in: Djidjev H. N., Pantziou G. E., Zaroliagis C. D. (1991) Computing shortest paths and distances in planar graphs. In: Albert J. L., Monien B., Artalejo M. R. (eds) Automata, Languages and Programming. ICALP 1991. Lecture Notes in Computer Science, vol 510. Springer, Berlin, Heidelberg, which is incorporated herein by reference. In another example, the similarity value is computed by

$$s i m ( {c}_{1} , {c}_{2} ) = \frac{2 i {c}_{l c s ( {c}_{1} , {c}_{2} )}}{i c ( {c}_{1} ) + i c ( {c}_{2} )}$$

where c₁ and c₂ are ontological concepts, lcs is the least common subsumer of c₁ and c₂ and ic is the information content of c as defined in Lin, D.: An Information-Theoretic Definition of Similarity. In: Proc. of Conf. on Machine Learning, pp. 296-304 (1998), which is incorporated herein by reference.

While Lin uses the Resnik model to compute the ic it may be preferable to instead use:

$$i c ( c ) = - \frac{log ( 1 + ( {c}_{leaves} / {c}_{a n c e s t o r s} ) )}{( {max}_{l e a v e s} + 1 )}$$

where cₗₑₐᵥₑₛ is the count of the leaf nodes under all children of c, c_{ancestors} is the count of all ancestors of c, maxₗₑₐᵥₑₛ is the total number of leaf nodes in the ontology. Further information can be found in Seco, N., Veale, T., Hayes, J. An Intrinsic Information Content Metric for Semantic Similarity in WordNet. Proceedings of the 16th European Conference on Artificial Intelligence, ECAI'2004 noting that Seco uses max_{nodes} instead of maxₗₑₐᵥₑₛ in their formula—i.e., the total count of nodes in the ontology.

Processor 104 repeats this calculation for each combination of observed phenotype with stored phenotype in the particular set so as to calculate a phenotype-to-phenotype similarity value indicative of a similarity between each of the observed phenotypes and each phenotype in the set of stored phenotypes, by determining a distance in the ontology from the observed phenotype to each phenotype in the set of stored phenotypes. For example, processor 107 may loop over all disorders in the database and for each disorder retrieve the set of phenotypes that define that disorder. Processor 104 may then perform a first loop over all stored phenotypes in that set and perform a second inner loop over the observed phenotypes and calculate the similarity value within the three nested loops (disorders, stored phenotypes and observed phenotypes).

Since this calculation can be relatively complex due to the large number of inner loops (combinations) the computation time can be reduced by splitting the phenotypes into the different anatomical systems, such that processor 104 never attempts to calculate a similarity value between phenotypes from different systems. For example, if there are 4,000 common diseases in the database with each having on average 8 phenotypes, there are 32,000 iterations in the first two loops. For 10 observed phenotypes this would result in 320,000 iterations in the innermost loop. Assuming 1,000 similarity measures can be determined per second, this would lead to 320 seconds (5 minutes) which is too long for a response user interface. Splitting the phenotypes into about 10 anatomical systems, for example, would mean that a large number of combination would not need to be calculated which would reduce the number of inner iterations in some examples by a factor of 10 to about 32 seconds which is more suitable for an entire rebuild of the disease database from scratch. It is a further advantage that the split along the top-level abnormalities (or anatomical systems) also keeps phenotypes localised—i.e., there would otherwise be a similarity value between large head (skeletal) and cafe-au-laix spots (skin), which, from a medical perspective, is not practical.

FIG. 4 illustrates a matrix 400 of similarity values for observed phenotypes 401 across stored phenotypes 402 for a first set. In this example, P1 denotes the first observed phenotype and P1,3 denotes the third stored phenotype of the first set (e.g. first disorder). Matrix 400 is basically a cost matrix and stores the distance in the number of nodes between the observed phenotypes and the stored phenotypes in the ontology. Zero values indicate that the observed phenotype is identical to the stored phenotype. Empty fields indicate a cost/distance above a threshold and the value is omitted simply for clarity of presentation. While the matrix 400 stores a cost as a similarity value, it is equally possible to store the closeness, such as a value of ‘1’ for identical nodes and progressively smaller values for nodes that are further apart. For example, the similarity value may be calculated as 1/(distance+1).

Once the phenotype-to-phenotype similarity values are calculated, processor 104 determines 205 an assignment of one stored phenotype of the set to each of the observed phenotypes based on the phenotype-to-phenotype similarity values. Fields with bold outlines indicate the assignment of a stored phenotype to an observed phenotype. As can be seen in FIG. 4, some observed phenotypes, such as P5 have two entries P1,6 and P1,8 and the assignment algorithm chooses the entry with the lowest distance. Assignment in this context means that there is a one-to-one relationship between a single observed phenotype and a single stored phenotype. It is noted that due to the split into anatomical systems, there is always a root node that connects any two phenotypes, which means there is also a possible path and therefore a finite cost in matrix 400. This also means that it should be possible in all circumstances to determine such one-to-one assignment. It is noted, however, that this assignment does not have to be optimal but a heuristic optimisation method may be sufficiently accurate with the advantage of a significant speed-up of calculation.

In one example, processor 104 performs the Hungarian algorithm described in Kuhn, H. W. (1955), The Hungarian method for the assignment problem. Naval Research Logistics, 2: 83-97, which is included herein by reference. The Hungarian algorithm works by first expanding the matrix to a square matrix, finding the minimum cost in each row and subtracting that cost from that row so as to generate one or more zero values. The same is then done for the rows. Processor 104 then determines a selection of zero values to cover the entire matrix by the minimum number of lines (rows or columns). If the number of selected rows/columns is less than the number of rows/columns of the matrix, processor 104 repeats the process. In this sense, processor 104 applies a heuristic to determine the assignment by selecting one assignment at a time with optimal cost and then determining remaining assignments. In one example, processor 104 executes code from the munkres Python module or the scipy.optimize.linear_sum_assignment Python module to determine the assignment.

FIG. 5 illustrates the result of the Hungarian assignment algorithm for the matrix in FIG. 4. As can be seen, seven stored phenotypes are assigned to seven observed phenotypes. Each of the assignments has a cost 500 associated with it which is the cost from the field in matrix 400 as a phenotype-to-phenotype similarity value. Again, the similarity value is the cost/distance in this example but other values may equally be used such as ‘1’ for direct matches and values between ‘1’ and ‘0’ for non-identical nodes.

Next, processor 104 aggregates 206 the phenotype-to-phenotype similarity values 500 of the stored phenotypes from the set that are assigned to each of the multiple observed phenotypes into a set-to-set similarity value indicative of a similarity between the observed phenotypes and the set of stored phenotypes. In this example, this aggregation comprises the calculation of an average value 501, which is ‘2.14’ in this example.

As mentioned above, processor 104 may split the observed phenotypes and stored phenotypes by anatomical systems and aggregating set-to-set similarity values across the anatomical systems. For example, P1, P2 and P3 may relate to the skeletal system, whereas P4, P5, P6 and P7 relate to the digestive system. In this case, the result of the assignment would be the same as before but the calculation to determines the assignment would be significantly reduced because the number of phenotypes in each set is reduced. In the example of split phenotypes, processor would calculate one average per system, that is, (0+2+5)/3=2.33 and (3+3+2+0)/4=2. Processor 104 can then aggregate the two results to calculate (2.33+2)/2=2.17. As can be seen, the difference between two methods is not significant but the reduction in computation time is significant.

While the above examples calculate averages, other aggregation methods may be used, such as sums, squared sums, etc. For example, processor 104 may simply sum up the cost values for the different systems into one sum and then divide by the number of phenotypes.

Processor 104 then repeats 207 the accessing 203, calculating 204, determining the assignment 205 and aggregating 206 steps for each of the multiple sets of stored phenotypes to thereby calculate a set-to-set similarity measure for each of the multiple sets. In other words, the processor 104 keeps the observed phenotypes for each iteration and calculates a set-to-set similarity between the set of observed phenotypes and each set of stored phenotypes, such as phenotypes defining disorders or being associated with other patients.

Once the set-to-set similarity values are calculated, processor 104 selects 208 one or more of the multiple sets based on the aggregated set-to-set similarity value. For example, processor 104 selects the highest ranked sets, such as the top 10 or top 4 sets or all sets that are above a threshold. This way, the number of sets (i.e. disorders) can be reduced from thousands to less than ten or less than five.

Processor 104 then generates 209 a graphical user interface comprising a graphical indication of the selected one or more of the multiple sets in relation to the multiple observed phenotypes. This may involve generating a user interface on a screen directly connected to computer system 100 where the processor 104 performs the calculations. It may also involve generating the user interface in the form of web-accessible content, such as HTML and JavaScript. Client 102 can then access the web-accessible content and render the graphical user interface on a screen of client device 102. Various different front-end/back-end platforms may be used including an Angular/Flask framework.

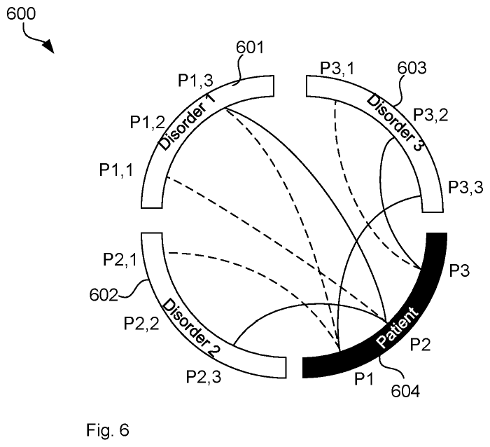

FIG. 6 illustrates an example user interface 600 comprising a graphical indication of a first set 601, a second set 602 and a third set 603. Again, these sets are the highest ranking sets out of the possibly large number of available sets. The graphical indications 601, 602 and 603 are placed in the user interface in relation to the multiple observed phenotypes 604. In the example of FIG. 6, processor 106 generates the graphical indications as annular segments so that the graphical indications of the disorders 601, 602 and 602 together with the observed phenotypes 604 form a ring. Since there are in total four segments, each segment occupies about a quarter of the ring (90 degrees). In case there are five segments (i.e. top four sets selected) each segment would occupy about a fifth of the ring (72 degrees).

User interface 600 also includes a graphical indication of the phenotype-to-phenotype similarities between the phenotypes in the selected sets 601, 602, 603 and the observed phenotypes 604. For example, processor 104 may generate a line between phenotypes that are similar. More particularly, processor 104 may generate a solid line between phenotypes that are an exact match (zero distance in the ontology graph) and dashed lines for inexact matches. There may be a threshold on the distance, such as 10, above which processor 104 draws no line.

Clinician 101 can now very clearly see which disorders are similar to the observed set of phenotypes and can also see which phenotypes are similar to understand the determined similarity. This means the method provides clinician 104 with guidance without taking control from the clinician's hands and without withholding or hiding important information from the clinician. In other words, the individual phenotypes are all displayed so that clinician 101 can make a professional conclusion but the data that is irrelevant is filtered out so as to provide a clear view on the data that is relevant.

While the above explanation and in particular FIG. 6 are related to disorders, the above method can equally be applied to other patients. That is, graphical indications 601, 602 and 603 may relate to first, second and third previously examined patients. Especially in cases where a large number of patients are in the database, such as more than 100, the method can reliably identify patients with similar disorders and clinician 101 can potentially consult with other clinicians that have diagnose or treated patients that are similar to the currently investigated patient.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for creating a graphical visualisation of clinical data, the method comprising:
receiving clinical data indicative of multiple observed phenotypes of a patient;
accessing a set of stored phenotypes of multiple sets of stored phenotypes;
accessing on a database an ontology of phenotypes including hierarchical relationships between the phenotypes of the ontology;
calculating, based on the ontology of phenotypes, a phenotype-to-phenotype similarity value indicative of a similarity between each of the multiple observed phenotypes and each phenotype in the set of stored phenotypes;
determining an assignment of one stored phenotype of the set to each of the multiple observed phenotypes based on the phenotype-to-phenotype similarity values;
aggregating the phenotype-to-phenotype similarity values of the stored phenotypes from the set that are assigned to each of the multiple observed phenotypes into a set-to-set similarity value indicative of a similarity between the multiple observed phenotypes and the set of stored phenotypes;
repeating the accessing, calculating, determining the assignment and aggregating steps for each of the multiple sets of stored phenotypes to thereby calculate a set-to-set similarity measure for each of the multiple sets;
selecting one or more of the multiple sets based on the set-to-set similarity values; and
generating a graphical user interface comprising a graphical indication of the selected one or more of the multiple sets in relation to the multiple observed phenotypes.

2. The method of claim 1, wherein determining the assignment comprises determining an assignment by optimising a cost that is based on the phenotype-to-phenotype similarity values.

3. The method of claim 2, wherein determining the assignment comprises applying a heuristic to determine the assignment by selecting one assignment at a time with optimal cost and then determining remaining assignments.

4. The method of claim 1, wherein determining the assignment comprises performing an Hungarian algorithm.

5. The method of claim 1, wherein aggregating the phenotype-to-phenotype similarity values comprises calculating an average of the phenotype-to-phenotype similarity values.

6. The method of claim 1, further comprising splitting observed phenotypes and stored phenotypes by anatomical systems and aggregating set-to-set similarity values across the anatomical systems.

7. The method of claim 6, wherein aggregating across the anatomical systems comprises calculating an average of the set-to-set similarity values the anatomical systems.

8. The method of claim 1, wherein generating the graphical user interface comprises generating a graphical indication of the phenotype-to-phenotype similarity values.

9. The method of claim 1, wherein the graphical indication of the phenotype-to-phenotype similarity value comprises a line with a first visual appearance for an exact match and a second visual appearance for an inexact match.

10. The method of claim 1, wherein the set of stored phenotypes is associated with a disorder.

11. The method of claim 1, wherein the set of stored phenotypes is associated with a further patient.

12. The method of claim 1, wherein calculating the phenotype-to-phenotype similarity value comprises determining a distance in the ontology from an observed phenotype to each phenotype in the set of stored phenotypes.

13. The method of claim 1, wherein calculating the phenotype-to-phenotype similarity value is based on a first information content of an observed phenotype in the ontology and an information content of the stored phenotype in the ontology and a second information content of a least common subsumer of the observed phenotype and the stored phenotype in the ontology.

14. The method of claim 13, wherein the first information content is based on a count of leaf nodes under children of the phenotype in the ontology, a count of ancestors of the phenotype in the ontology and a total number of leaf nodes in the ontology.

15. A computer system for creating a graphical visualisation of clinical data, the computer system comprising:
a data port to receive clinical data indicative of multiple observed phenotypes of a patient;
a data store from which to access a set of stored phenotypes of multiple sets of stored phenotypes;
database to store an ontology of phenotypes including hierarchical relationships between the phenotypes of the ontology;
a processor to:
calculate, based on the ontology of phenotypes, a phenotype-to-phenotype similarity value indicative of a similarity between each of the multiple observed phenotypes and each phenotype in the set of stored phenotypes;
determine an assignment of one stored phenotype of the set to each of the multiple observed phenotypes based on the phenotype-to-phenotype similarity values;
aggregate the phenotype-to-phenotype similarity values of the stored phenotypes from the set that are assigned to each of the multiple observed phenotypes into a set-to-set similarity value indicative of a similarity between the multiple observed phenotypes and the set of stored phenotypes;
repeat the accessing, calculating, determining the assignment, and aggregating steps for each of the multiple sets of stored phenotypes to thereby calculate a set-to-set similarity measure for each of the multiple sets;
select one or more of the multiple sets based on the set-to-set similarity values; and
generate a graphical user interface comprising a graphical indication of the selected one or more of the multiple sets in relation to the multiple observed phenotypes.

16. A non-volatile computer-readable medium with software code stored thereon that, when executed by a computer, causes the computer to perform one or more actions comprising:
receiving clinical data indicative of multiple observed phenotypes of a patient;
accessing a set of stored phenotypes of multiple sets of stored phenotypes;
accessing on a database an ontology of phenotypes including hierarchical relationships between the phenotypes of the ontology;
calculating, based on the ontology of phenotypes, a phenotype-to-phenotype similarity value indicative of a similarity between each of the multiple observed phenotypes and each phenotype in the set of stored phenotypes;
determining an assignment of one stored phenotype of the set to each of the multiple observed phenotypes based on the phenotype-to-phenotype similarity values;
aggregating the phenotype-to-phenotype similarity values of the stored phenotypes from the set that are assigned to each of the multiple observed phenotypes into a set-to-set similarity value indicative of a similarity between the multiple observed phenotypes and the set of stored phenotypes;
repeating the accessing, calculating, determining the assignment, and aggregating steps for each of the multiple sets of stored phenotypes to thereby calculate a set-to-set similarity measure for each of the multiple sets;
selecting one or more of the multiple sets based on the set-to-set similarity values; and
generating a graphical user interface comprising a graphical indication of the selected one or more of the multiple sets in relation to the multiple observed phenotypes.
